(19) United States
(12) Patent Application Publication
(10) Pub. No.: US 2004/0127892 A1
(43) Pub. Date: Jul. 1, 2004

(54) Periodontal laser and methods

(76) Inventor: David M. Harris, Castro Valley, CA (US)

Correspondence Address: HAVERSTOCK & OWENS LLP, 162 NORTH WOLFE ROAD, SUNNYVALE, CA, 94086, US

(21) Appl. No.: 10/661,261

(22) Filed: Sep. 11, 2003

Related U.S. Application Data
(63) Continuation-in-part of application No. 10/066,162, filed on Jan. 31, 2002.
(60) Provisional application No. 60/410,488, filed on Sep. 12, 2002.
(60) Provisional application No. 60/464,929, filed on Apr. 22, 2003.

(51) Int. Cl. A61B 18/20

(52) U.S. Cl. 606/11; 606/12; 606/15; 606/16

(57) ABSTRACT

An apparatus for and method of developing laser therapies to eradicate or ablate one or more pathogens in periodontal tissues are disclosed. The apparatus comprises an adjustable laser source, that is preferably a Nd:YAG laser, for directing pulsed laser light to an area of a target comprising the one or more pathogens, such as phorphyromonas gingivalis (Pg) and prevotella intermedia (Pi) and/or a pigment fungi. The apparatus preferably comprises means for measuring the laser power provided to the target from the adjustable laser source and means for determining if exposed pathogens within the target have been eradicated or ablated. By identifying damage thresholds of laser doses, therapeutic protocols for treating periodontal tissues infected with the one or more pathogens are developed.

### RELATED APPLICATION(S)

This Patent Application is a continuation-in-part of U.S. patent application Ser. No. 10/066,162, filed Jan. 31, 2002, and titled “METHOD OF PERIODONTAL LASER TREATMENT.” This patent application also claims priority under 35 U.S.C. §119 (e) of the co-pending U.S. Provisional Patent Application, Serial No. 60/410,488, filed Sep. 12, 2002, and titled “PULSED Nd:YAG LASER SYSTEM” and the co-pending U.S. Provisional Patent Application, Ser. No. 60/464,929, filed Apr. 22, 2003, and titled “METHOD OF PERIODONTAL LASER TREATMENT.” The co-pending U.S. patent application Ser. No. 10/066,162, filed Jan. 31, 2002, and titled “METHOD OF PERIODONTAL LASER TREATMENT”; the co-pending U.S. Provisional Patent Application, Serial No. 60/410,488, filed Sep. 12, 2002, and titled “PULSED Nd:YAG LASER SYSTEM”; and the co-pending U.S. Provisional Patent Application, Serial No. 60/464,929, filed Apr. 22, 2003, and titled “METHOD OF PERIODONTAL LASER TREATMENT” are all hereby incorporated by reference.

### BACKGROUND OF THE INVENTION

Gum disease, or periodontal disease, is commonly associated with the presence of bacterial pathogens within periodontal pockets between the dentin and gum tissue. Gingivitis describes a periodontal condition of inflammation within the superficial layers of the periodontium. Periodontitis is advanced Gingivitis, whereby the inflammation is extended to the underlying tooth supporting structures and other deep periodontal tissues. Attachment loss and gum recession is symptomatic of advance gingivitis or periodontitis which can leave extremely sensitive portions of underlying tooth supporting structures exposed.

Ultimately, periodontitis leads to the destruction of both supra-alveolar and periodontal fibers as well as the adjacent portion of the alveolar bone which generally provides for the attachment of healthy soft periodontal tissue to the cementum. When the soft periodontal tissue becomes inflamed as a result of bacteria, the edematous and junctional epithelium recedes away from the cementum creating an enlarged periodontal pocket and attachment loss of the soft periodontal tissue to the cementum.

Enlarged periodontal pockets provide collection sites for plaque and calculus which adheres to root surfaces. Calcified plaque and calculus provide rough surfaces which are a highly suitable environment for hosting and colonizing bacterial pathogens. One category of bacterial pathogens which has been strongly implicated in the progression of periodontal disease are referred to as gram (-) anaerobic pathogenic bacteria.

Periodontal bacteria have been shown to migrate into surrounding soft tissues and survive within endothelial cells, macrophage and perivascular cells. Periodontal bacteria are also can survive in hard periodontal tissue including dentin, bone and cementum tissue. Periodontal bacteria can also enter into the general circulation system through various systemic routes and mechanisms.

Periodontal disease has been correlated to several systemic conditions, such as cardiovascular disease, and is thought to contribute to other heath problems including pre-term delivery and low infant birth weight for infants delivered from mothers having periodontal disease. While there is no comprehensive list of health related problems associated with periodontal disease nor is there a complete understanding as to whether periodontal disease is aggravated by other health conditions or vise-versa, it is commonly believed that periodontal disease can propose a health risk.

The most effective therapy for gum disease is to motivate patients to improve their personal oral hygiene habits. Unfortunately, attachment loss is a progressive condition due to the activation mechanisms of collagen destruction and bone resorption. Therefore, adhering to a aggressive maintenance regimen will not typically improve the gum recession. However, gum recession can be stopped, or at least the rate of gum recession can be significantly reduced.

Topological antibiotic therapies are usually ineffective in the eradication of periodontal bacteria because a portion of the bacteria which survive such therapies and can re-colonize. Bacteria survive topological antibiotic therapies by virtue of being isolated deep within dental calculus and/or intracellular locations of periodontal tissues and are, therefore, topologically privileged. Systemic antibiotic therapies are also typically ineffective in the eradication of periodontal bacteria because dental calculus and intracellular locations are also systemically privileged and not accessible by the host's circulation system. Besides being ineffective, antibiotic therapies can lysis non-privileged bacteria causing fragments of the bacteria to enter the blood stream and result in “endotoxic shock” or “septic shock” to the patient.

Because of the numerous shortcomings of antibiotic therapies, mechanical methods are usually employed, either solely or in combination with antibiotic therapies. Mechanical procedures, also referred to herein as debridement, include removing calculus, diseased cementum and/or necrotic soft tissue within the gingival sulcus containing the bacteria. These procedures are more commonly referred to as scaling, root planing and sulcular debridement. In debridement procedures, a curette, ultrasonic scalar or any other suitable device is used to remove infected or diseased tissue from healthy tissue with the intent of reestablishing attachment of the remaining healthy soft periodontal tissue.

There are even more aggressive treatments for patients with highly advanced periodontitis. In these more aggressive treatments flaps from the gum tissue are cut and removed or pulled away from the root structures in order to access the root surface so that the diseased tissues can be removed. After the diseased tissues are removed, the gingival flaps are sutured back into place. Grafting procedures are also frequently used to “build-up critical tissues” around dentition, wherein the critical tissues have been depleted from periodontitis or treatments thereof.

In addition to the obvious discomfort suffered to the patient during and after these aggressive mechanical treatments, such mechanical treatments also have several shortcomings. Mechanical treatments are not a cure for periodontitis because pathogen may survive within the periodontal tissues. Even a small amount of living bacteria within the soft tissues, hard tissues or semi-hard tissues can allow the bacteria to re-colonize quickly after the treatment. Mechanical treatments can result in the systemic release of toxic bacteria fragments leading to toxic shock and possible other health problems. Also, mechanical treatments can only be implemented a limited number of times without requiring oral surgery and/or grafting of gum tissues. Some patients are, unfortunately, highly susceptible to periodontal disease and debridement is not a viable long term solution to prevent the advancement of attachment loss of the soft periodontal tissue to the cementum.

What is needed is a system for and method of treating pathogens within an oral cavity. Further, what is needed is periodontal treatment which can be used to treat periodontal tissues in the early phases of periodontal disease and which can be administered multiple times without causing serious trauma to gum tissue and without causing significant systemic release of toxins from treated pathogens.

### SUMMARY OF THE INVENTION

The current invention provides a system for and method of generating at least one antiseptic pulse to a target area of a target tissue. The target tissue is preferably a periodontal tissue. The antiseptic pulse is preferably a high-energy pulse of laser light which is preferentially absorbed by one or more target pathogens. The high-energy pulse of laser light preferably penetrates into the target periodontal tissue to a distance of 1.0 mm or greater and eradicates the target pathogens, or a portion thereof, within the target tissue. The depth to which the pathogens are eradicated is referred to, herein, as the effective treatment depth. The effective treatment depth multiplied by the target area exposed by each laser pulse is referred to, herein, as the effective treatment volume. The method of the instant invention preferably utilizes laser pulses which denature or coagulate the target pathogens within the effective treatment volume and is, therefore, coined as photo-thermo-coagulation.

Unlike with antibiotic therapies, periodontal bacteria will not likely develop resistance to photo-thermo-coagulation. Further, because periodontal tissues are substantially transparent to the laser radiation used, the treatment can be administered a number of times without significantly effecting healthy periodontal tissues. Also, it is believed that photo-thermo-coagulation does not result in significant systemic release of toxins. The periodontal laser treatment, described below, can be used in conjunction with debridement procedures and antibiotic therapies. However, the periodontal laser treatment of the instant invention is preferably used in place of aggressive mechanical treatments.

The target pathogens can be any number of pathogens that selectively absorb the laser pulses used, and which preferably are implicated in periodontal disease. Bacteria which are known to cause periodontal disease, include but are not limited to, pigmented gram (-) anaerobes such as phorphyromonas gingivalis (Pg) and prevotella intermedia (Pi). The current invention can also be used to treat pigmented fungus such as Histoplasma and Aspirgillus Niger.

In accordance with the method of the instant invention, the target pathogens are preferably located within the oral cavity. In advanced gingivitis, observation of the soft periodontal tissue may be sufficient to diagnose for the presence and the location of the target pathogens. In early stages of periodontal disease, however, a culture or the use of other analytical techniques (such as a DNA testing) may be required to identify or locate the target pathogens.

The periodontal laser treatment may be tailored to a specific target pathogen by spectroscopically characterizing the specific target pathogen and selecting a laser treatment wavelength corresponding to a larger absorption coefficient for the specific target pathogen. Preferably, the absorption coefficient of the specific target pathogen is at least ten times greater than the absorption coefficient of normal or healthy periodontal tissue.

After the pathogen has been located and/or characterized within the oral cavity, an area of target tissue is irradiated with at least one pulse of laser light. In accordance with an embodiment of the instant invention, the target tissue is soft periodontal tissue surrounding a periodontal pocket. In further embodiments of the instant invention, the target tissue comprises cementum, dentin and or infected bone. Accordingly, the pulse of laser light can be delivered external to the periodontal pocket or from within the periodontal pocket by placing a firing end of an optical fiber near the target tissue and pulsing laser light through the optical fiber.

The pathogen is preferably irradiated with one or more pulses of laser light each having sufficient energy to eradicate the target pathogen, or a portion thereof, within the effective treatment volume. The laser treatment is preferably 1.0 second, or less, of laser radiation to each target area treated. When the target area is outer soft periodontal tissue, the laser radiation preferably, penetrates a distance of 1.0 mm or greater into the soft periodontal tissue. More preferably, the laser radiation penetrates 2.0 mm or greater into the soft periodontal tissue, such that a portion of the pathogens within the periodontal pocket and the inner pocket tissues are eradicated without requiring mechanical displacement of the patient's gums.

When the laser source is a Nd:YAG laser source, laser pulses preferably have energy concentrations of 10 J/cm2 or greater within the tissue(s) at the site of pathogen eradication. However, the specific dosimetry that is chosen is dependent on the optical properties of the irradiated tissue(s) including, but not limited to, transmission through non-target tissues and absorption coefficients of target tissues at the light source wavelength. Preferably, laser radiation is delivered to each area treated with a laser fluence that is sufficient or eradicate pathogens within or on the target tissue with minimal damage to the healthy tissue. Tissues can ablate at approximately 400 Joule/cm². Accordingly, for many of the applications described herein it is preferable that the laser radiation is delivered to each area treated with a laser fluence is 350 Joule/cm² or less and more preferably with a laser fluence of around 50 Joule/cm². The actual laser fluence chosen, will depend on the specific application and on the pathogen or pathogens being targeted.

Prior to treating target pathogens with pulsed laser light, the pathogens can be stained with a staining agent or a pigment to facilitate the absorption of the laser light. Also, prior to the laser treatment or, alternatively, after the laser treatment, the pathogens can be stained with a staining agent which stains for the presence of living bacteria. The periodontal laser treatment can be administered any number or times as required to eradicate the bacteria or until the observed concentrations of the living bacteria are at prescribed levels.

A laser system in accordance with the instant invention, is configured to deliver pulses of laser radiation to target periodontal tissues within an oral cavity. The laser system comprises a laser source, that is preferably a pulsed laser source, for generating pulsed laser light having a peak wavelength in a range of 600 to 1100 nanometers. The system preferably includes a delivery system with an arm structure and an applicator.

The delivery system is configured with any suitable optics for delivering the laser light from the laser source to the target tissues. Suitable optics include, but are not limited to lenses, mirrors, optical fibers and scanning mirrors. Preferably, the delivery system is configured to deliver pulsed laser light to a wide field exposure area. The wide field exposure area preferably corresponds to a surface area of 1.0 to 9 mm² or more.

In accordance with an embodiment of the invention, the applicator includes a side-firing optical fiber configured to emit pulsed laser light at an angle from the distal or firing end of the optical fiber. The applicator can also be configured with a soft resilient guide member for controlling a distance between the laser light emitted from the distal or firing end of the optical fiber and the target tissue. Preferably, the present invention utilizes an optical fiber hand piece with a removable and/or disposable endo-probe for single use application of laser radiation periodontal tissue.

The present invention is also directed to an apparatus for determining an antiseptic laser dose. The apparatus comprises an adjustable laser source for generating a laser output. The adjustable laser source is preferably configured to provide a range of laser exposures to a target. The exposures can be varied by changing the laser output power from a delivery optical fiber of the laser source, changing the exposure area (the laser spot size), changing a distance between the delivery optical fiber of the laser source and the target or any combination thereof. The distance between the delivery optical fiber of and the target can be controlled with a stepper motor unit that is configured to incrementally change the distance between the delivery optical fiber and the target.

The apparatus, in accordance with further embodiments of invention, comprises means for holding the target in a path of the laser output. The means for holding the target in the path of the laser output can include a stand and dish configured for holding the target. The target can include a medium, such as gelatin, and a pathogen therein, such as phorphyromonas gingivalis (Pg) and prevotella intermedia (Pi) and/or a pigment fungi that has been cultured. Methods of growing cultures are described in the U.S. Provisional Patent Application, Serial No. 60/464,929, filed Apr. 22, 2003, and titled “METHOD OF PERIODONTAL LASER TREATMENT” referenced previously.

The apparatus preferably further comprises a means for measuring the laser output power delivered to the target from an adjustable laser source. For example, the means for measuring the laser output power comprises a power meter that is configured to be positioned behind the target and in the path of the laser output.

In accordance with still further embodiments of the invention, the apparatus comprises an optical detector, such as an optical microscope, an optical scanner, a video camera or any combination thereof. The optical detector is permeably configured for determining when and if exposed pathogens within the target have been eradicated or ablated by the laser exposure and/or determines the percentage of exposed pathogens that are eradicated or ablated by the laser exposure. The apparatus can also include an audio detector for detecting clicks or sounds generated while exposing the target with the laser radiation.

The apparatus, in accordance with further embodiments of the invention, comprises a computing means. The computing means is coupled to the laser source, the power meter, the optical detector, the stepper motor or any combination thereof. The computing means can, in accordance with the embodiments of the invention, be interfaced with the laser source, the power meter, the optical detector, the stepper motor unit or any combination thereof, to automatically collect and store data acquired during exposure of the target with laser radiation. Data that are collected and stored can include laser output power levels, exposure times, exposure spot sizes, laser repetition rates, percentages of pathogens ablated, and distances between the laser output and the target during an exposure sequence. Accordingly, multiple pathogen targets and/or samples can be treated under a wide range of laser exposure conditions to provide statistically significant data that can then be used to develop laser treatment protocols for treating periodontal tissue infected, for example, with the pathogens.

In accordance with the method of the present invention, an antiseptic dose for eradicating or ablating pathogens in a target is determined by measuring the power of a pulsed laser output from a laser source. After measuring the output power of the pulsed laser, regions of the target comprising the pathogen are exposed with the pulsed laser output. The exposed regions of the target are then examined to determine if the pathogens within the exposed regions of the target have been eradicated or ablated. If a significant percentage (75 percentage or more) of the pathogens have been eradicated or ablated by laser exposure, then the output power of the laser source is reduced and the exposure steps are repeated in a new region of the target. If a significant percentage of the pathogens have not been eradicated or ablated in the exposed area of the target, then the laser output power is increased and the exposure steps are repeated in a new region of the target. By an iterative process, such as described above, a range of exposure conditions that are capable of eradicating or ablating pathogens can be determined. It should be noted that, as described below, the repetition rate or the pulse frequency of the laser source can influence the effectiveness of the laser output to eradicate or ablate pathogens in the exposed regions of the target. In fact, it is believed that the effectiveness of the laser output to eradicate or ablate pathogens in exposed regions of the target can be enhanced by pulsing the laser source at a photo-acoustic frequency corresponding to the target, wherein the energy can build up within the target with each subsequent pulse of the laser source. After the damage, therapeutic or antiseptic dose for a pathogen has determined, then the knowledge can be used to develop of tissue responses to laser exposures, therapeutic protocols or models for treating periodontal tissues infected with the pathogens can be developed.

### BRIEF DESCRIPTION OF THE DRAWINGS

FIG. 1 illustrates a schematic representation of human dentition.

FIG. 2A illustrates a cross-section of a periodontal pocket and a method of applying laser radiation to periodontal tissues from within the periodontal pocket, in accordance with the current invention.

FIG. 2B illustrates a cross-section of a periodontal pocket and methods of applying laser radiation to the periodontal pocket through tissues outside of the periodontal pocket, in accordance with the current invention.

FIG. 3 shows absorption spectra of water, hemoglobin, and melanin between 0.1 and 10 micron wavelength light.

FIG. 4A illustrates applying laser radiation to the outer soft periodontal tissue from a delivery system, in accordance with the instant invention.

FIG. 4B illustrates an endo-probe with a side-firing optical fiber for treating periodontal tissues, in accordance with the embodiments of the invention.

FIGS. 4C-E show delivery optical fiber configurations for treating periodontal tissues, in accordance with the embodiments of the invention.

FIG. 5 is a schematic representation of the laser system for treating periodontal tissue with laser radiation, in accordance with the instant invention.

FIGS. 6A-B show views of an applicator portion having an optical fiber and guide member for treating periodontal tissue, in accordance with the current invention.

FIG. 7A shows an apparatus for measuring therapeutic or antiseptic laser dose for eradicating or ablating pathogen in a target, in accordance with the embodiments of the invention.

FIG. 7B shows a block diagram outlining steps for determining a therapeutic or antiseptic laser dose for eradicating or ablating pathogens in a target, in accordance with the method of the present invention.

### DETAILED DESCRIPTION OF THE PREFERRED EMBODIMENT

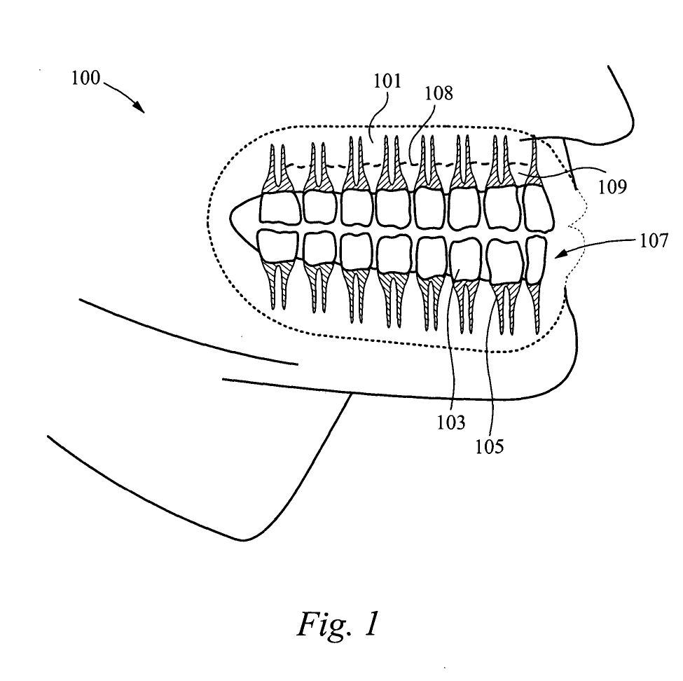

FIG. 1 illustrates a schematic representation of human dentition 100. Teeth 103 in an oral cavity 107 are embedded within surrounding periodontal tissue 101. The teeth 103 are attached to a jaw bone (not shown) through root structures 105. In advanced cases of periodontal disease, the periodontal tissue 101 detaches from portions of the root structures 105, as indicated by the dotted lines 108, thereby forming periodontal pockets and/or exposing sensitive portions 109 of the root structures 105. Late stages of periodontal disease and result in the complete detachment of the periodontal tissue 101 from the underlying root structures 105 and ultimately result in the loss of teeth 103, as described below.

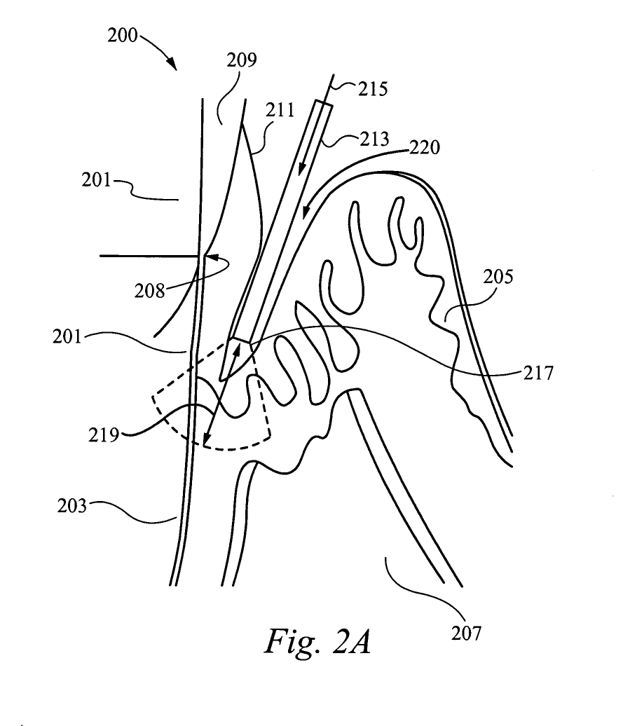

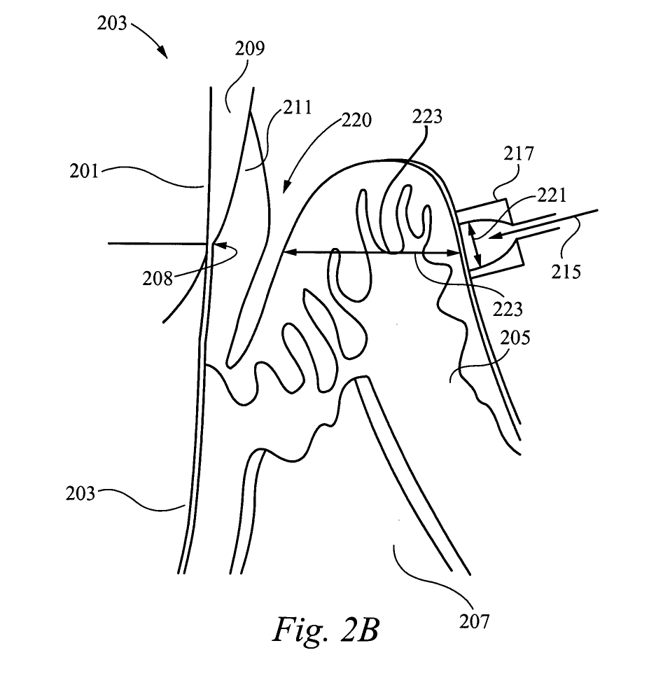

FIG. 2A shows a cross-sectional view 200 of a periodontal pocket 220 and surrounding periodontal tissues. With periodontal disease, the soft periodontal tissue 205 become detached or separated from the cementum 203, which is a layer of hard tissue on the outer surface of the root. Below the cementum 203 is the dentin 201. Normally, the periodontal ligament(s) attach up to cemento-enamel junction 208. As periodontal disease progresses, this attachment level recedes apically (toward the root). The ultimate result can be loss of attachment to the bone 207 an loss of the tooth.

When the soft periodontal tissue 205 remains above the point of attachment, then a periodontal pocket 220 is formed. For example, the attachment loss, as measured from the cemento-junction 208 is 8.0 mm. If the soft periodontal tissue 205 has receded apically by 3.0 mm, then the depth of the periodontal pocket 220 is 5.0 mm.

The calculus 211 and plaque within the periodontal pocket 220 are, unfortunately, excellent hosts for bacterial growth which can lead to advanced periodontal disease, as described above. The bacteria which is colonized within the periodontal pocket 220 can penetrate surrounding periodontal tissues and occupy intercellular positions within the soft periodontal tissues 205, the bone tissue 207 and the cementum 203 making topological or systemic antibiotic therapies ineffective for the eradication of the bacteria and/or fungus.

Accordingly, the current invention seeks to eradicate such pathogens within the periodontal pocket 220 and within the surrounding tissues by providing an antiseptic laser pulse or sequence of antiseptic laser pulses having a wavelength in the range of 600 to 1100 nanometers. The laser pulses are generated from any suitable laser source including a Nd:YAG laser source, a solid-state laser diode, a gas laser source or combinations thereof. The laser radiation from the laser pulses, preferably penetrate the soft periodontal tissue 205 by a distance 219 of at least 1.0 mm and more preferably by a distance 219 of at least 2.0 mm.

The laser treatment can be less than 1.0 second for each area treated, but preferably each area treated is exposed to laser radiation with energy concentrations of at least 17.0 J/cm², a laser fluence of at least 350 Joule/cm² and total energy of at least 2 Joules in order to ensure that target pathogens within the effective treatment volume are eradicated.

Still referring to FIG. 2A, in order to administer the laser radiation 215 to the target tissue, an optical fiber 213 is inserted in the periodontal pocket 220. The laser radiation 215 is delivered through the firing end 217 of the optical fiber 213. The optical fiber 213 can be moved up and down or side-to-side within the periodontal pocket 220 to ensure that the entire periodontal pocket 200 is treated with the laser radiation.

FIG. 2B illustrates a cross-section 203 of the periodontal pocket 220 along with the surrounding dentin 201, cementum 203, bone 207, enamel 209 and soft periodontal tissue 205. Again, the soft periodontal tissue 205 is slightly separated from the cementum 203 and/or dentin 201 as a result of infectious bacteria colonized on or within the calculus 211, calcified plaque and/or surrounding tissues.

In accordance with the instant invention, laser radiation is delivered to a target tissue with penetration depth of 2.0 cm and less, wherein the surface is treated with antiseptic laser pulses as well as the tissue(s) below the surface. Soft periodontal tissues, hard periodontal tissues and plaque can be treated in accordance with the instant invention to phot-coagulate host pathogens In accordance with an alternative embodiment of the invention, laser radiation 215 is delivered from a laser applicator to outer portions of the soft periodontal tissue 205. Accordingly, the laser applicator 217 is placed onto or next to the outer portions of the soft periodontal tissues 205 and at least one antiseptic laser pulse is delivered to the target area of the soft periodontal tissue 205. Preferably, the target area is a wide field exposure area 217 corresponding to a surface area of 1.0 to 9 mm², or greater. The laser radiation 215, or a portion thereof, preferably penetrates to a depth 223 of 2.0 cm or less through soft periodontal tissue 205, dentin 201, cementum 203 bone tissue 207 or any other infected tissue, such that at least a portion of the target pathogens within the periodontal pocket 220 and or/within the target tissues are eradicated. Preferably, the laser treatment also effectively eradicates both intra- and extra-cellular pathogens within the irradiated sort tissue 205, hard tissues 201, 203 and 207 and/or the plaque

In a preferred embodiment the dosimetry of the laser treatment corresponds to an effective treatment volume of laser pulses, wherein the pathogen within the treatment volume of the are substantially irradiated.

The larger field exposure area 211 can be irradiated through an optical fiber, a focusing lens, a bundle optical fibers or any combination thereof. Alternatively, a large field exposure area 211 is irradiated by a laser scanner which either rasters the laser beam 215 over the target area or, alternatively, projects a series of closely spaced or overlapping spots onto the target area.

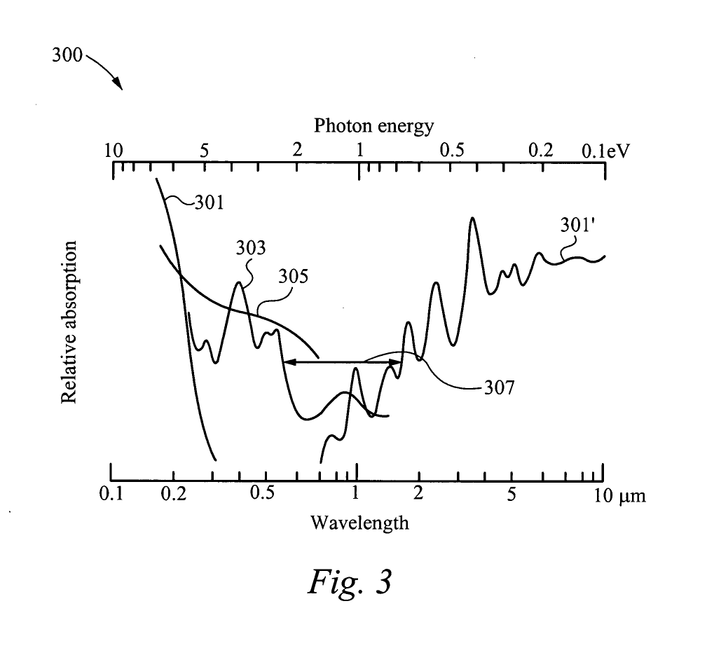

FIG. 3 shows absorption spectra 300 for water, hemoglobin and melanin, which are major contributors to the absorption of light in periodontal tissues. The lines 301 and 301′ in the graph of FIG. 3 correspond to the absorption of water, the line 303 corresponds to the absorption of hemoglobin and the line 305 corresponds to the absorption of melanin for light having wavelengths between 100 to 10,000 nanometers. The absorption spectra 300 shows that there is a preferred window of wavelengths 307 which are essentially non-absorbing water, hemoglobin and melanin corresponding approximately to light having wavelengths between 550 to 1800 nanometers.

In developing a selective treatment for a target pathogen or target pathogens, the target pathogens are isolated from target tissues. The pathogens are then preferably characterized by collecting absorption spectra for one or more of the target pathogens within the preferred wavelength window 307. After the pathogens have been spectroscopically characterized within the preferred wavelength window 307, then a laser treatment wavelength is selected which corresponds approximately to an absorption peak of at least one of the target pathogens. When a mixture of pathogens is present, the laser treatment wavelength can be chosen such as to maximize the collective absorptions of each pathogen within the mixture of target pathogens. Alternatively, different treatment procedures can be developed for each pathogen or for a selected group of pathogens within the mixture of target pathogens.

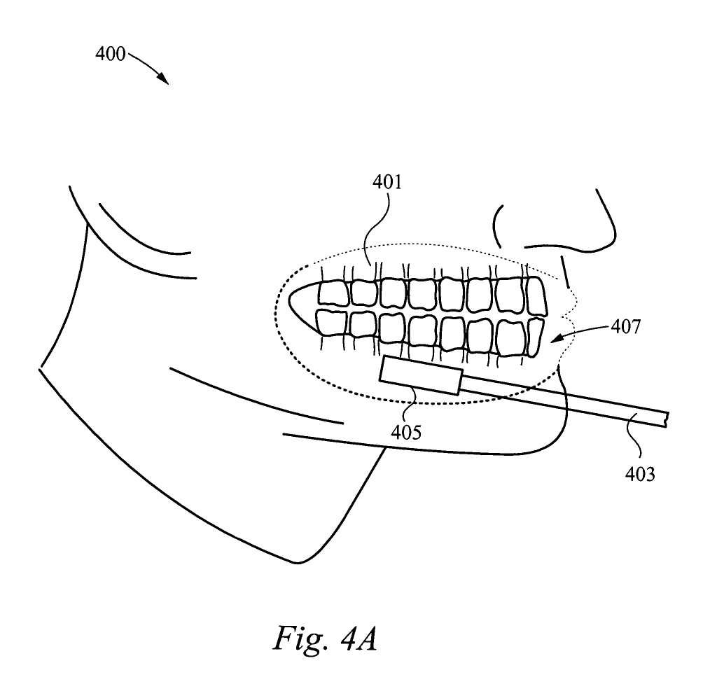

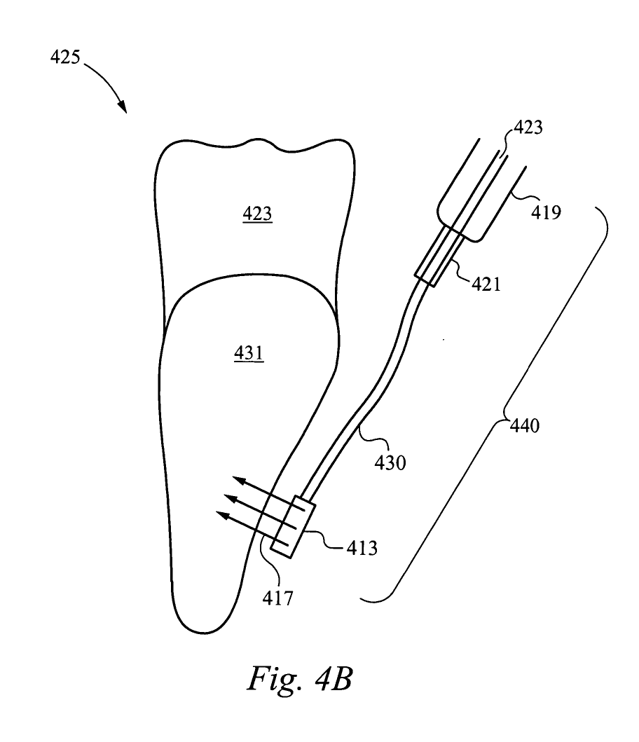

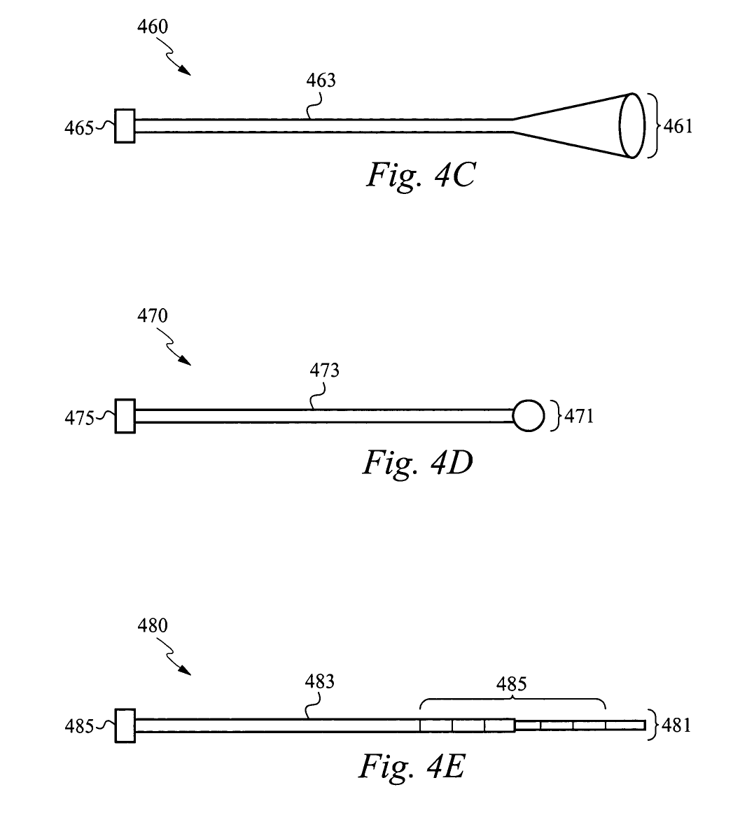

Now referring to FIG. 4A, the laser radiation is preferably applied to the soft periodontal tissue 401 through a laser delivery system comprising an arm feature 403 and an applicator portion 405. Target pathogens within the oral cavity 407 can be stained with a staining agent which facilitates the absorption of the laser radiation by the pathogens. The arm feature 403 can house one or more optical fibers. Alternatively, the arm feature is a jointed arm with a series of focusing lens and/or mirrors for focusing a laser beam through the applicator 405 and onto the soft periodontal tissue 401. The choice of delivery system used depends in-part on the type of laser source used, because certain laser wavelengths are absorbed by typical optical fibers making such delivery systems less preferable.

In still further embodiments, the laser source is within the arm feature 403 or within the applicator portion 405. For example, the laser source is a high-powered laser diode that is housed within the arm feature 403 or applicator 405 and coupled with the appropriate optics to deliver laser radiation onto the soft periodontal tissue 401 and/or teeth dentin, cementum, bone plaque or any other infected periodontal tissue, as described preciously.

After the laser treatment of the soft periodontal tissue 401, as described above, the periodontal tissues within the oral cavity 407 can be tested for the presence of the target pathogens. The periodontal tissues within the oral cavity 407 can be tested for the presence of the target pathogens by growing a culture or by staining techniques, wherein topographically accessible pathogens are stained with a staining agent or pigment which stains for the presence of living pathogens. In the event that a number of pathogens are still present, then a second laser treatment can be administered to the soft periodontal tissue 401. Periodontal tissues within the oral cavity 407 can also be subjected to mechanical debridement procedures if necessary and/or antibiotic treatments prior to, during or after the laser treatment. Applying the laser treatment prior to mechanical or debridement procedures helps to decreases the concentration of pathogen or endotoxins in the debris generated form scaling, planing and secular debridement, and subsequently reduces the potential for the release of pathogen or endotoxins in to the patient's saliva and/or circulation system. However, the laser treatment described above, is preferentially used in place of aggressive mechanical tenements.

FIG. 4B illustrates a simplified and schematic representation of tooth structure 425 with exposed bone tissue 423 and root bone tissue 431. Soft periodontal tissues and other tissue structures have been eliminated for clarity. However, it is understood that such tissues and structures are present during treatment with laser radiation. Preferably, periodontal tissues and/or structures are treated with an antiseptic dose of laser radiation which eradicates pathogens, such as those described above, with a minimal damage to healthy periodontal tissues and structures.

In accordance with a preferred embodiment of the invention, the antiseptic dose of laser radiation is delivered to periodontal tissues and/or structure using an optical fiber applicator 440. The optical fiber applicator 440 preferably comprises a handle section 419, a trunk fiber 423 therein, a delivery optical fiber 430 and an optical fiber coupler 421 for couple the delivery optical fiber 430 to the trunk fiber 423. The trunk fiber 423 is coupling to a laser output source (not shown), which is preferably a Nd:YAG laser output source.

Still referring to FIG. 4B, the delivery optical fiber 430 is preferably a side firing optical fiber that can be placed under soft periodontal tissue or between soft periodontal tissue and/or structures to deliver the laser radiation 417. The firing head 413 of the side firing optical fiber 430 can be modified in a number of different ways to broaden or narrow the distribution of the laser radiation 417 delivered to periodontal tissues and/or structures. Preferably, the delivery optical fiber 430 is configured to be readily detached from the handle section 419 and replaced with a new delivery optical fiber after a single use. An number of other delivery optical fiber constructions have ben contemplated, a few of which are briefly described below.

FIGS. 4C-E show delivery optical fiber configurations for treating periodontal tissues, in accordance with the embodiments of the invention. Referring now to FIG. 4C, the delivery optical fiber 460 comprises a body 463 with an expanded distal or firing end 461. The expanded distal or firing end 461 is preferably configured to deliver low power density laser radiation to a surface of target tissue during a laser exposure, but is preferably configured to provide a sufficient power density laser radiation to the exposed target tissue so as to penetrate into the target tissue and eradicate or ablate pathogens therein. FIG. 4D illustrates a delivery optical fiber 470 with a body 473 and a bulbous or rounded distal or firing end 471. The bulbous or rounded distal or firing end 471 is preferably configured to deliver low power density laser radiation or dispersed laser radiation to a surface of a target tissue during a laser exposure, but is preferably configured to provide a sufficient power density laser radiation to the exposed target tissue, so as to penetrate into the target tissue and eradicate or ablate pathogens therein. The bulbous or rounded distal or firing end 471 of the delivery optical fiber 470 can be configured to insert into a periodontal pocket during laser treatment. Now referring to FIG. 4E, which shows a delivery optical fiber 480 with a body 483 with a truncated distal or firing end 481. The truncated distal or firing end 481 of the delivery optical fiber 480 is preferably configured to insert into a periodontal pocket during laser treatment and eradicate or ablate pathogens therein In accordance with a preferred embodiment of the invention, the delivery optical fiber 480 has gradations or markings 485 on the body 483 or the truncated distal or firing end 481 of the delivery optical fiber 480 for measuring depths of the periodontal pockets while simultaneously treating the periodontal pockets to laser radiation.

Still referring to FIGS. 4C-4E, the delivery optical fibers 460, 470 and 480 preferably are configured to be detachable from the rest of a laser delivery system and comprise an optical coupling means 465, 475 and 485, respectively, for coupling the delivery optical fibers 460, 470 and 480 to a laser source, such as described with reference to FIG. 4B.

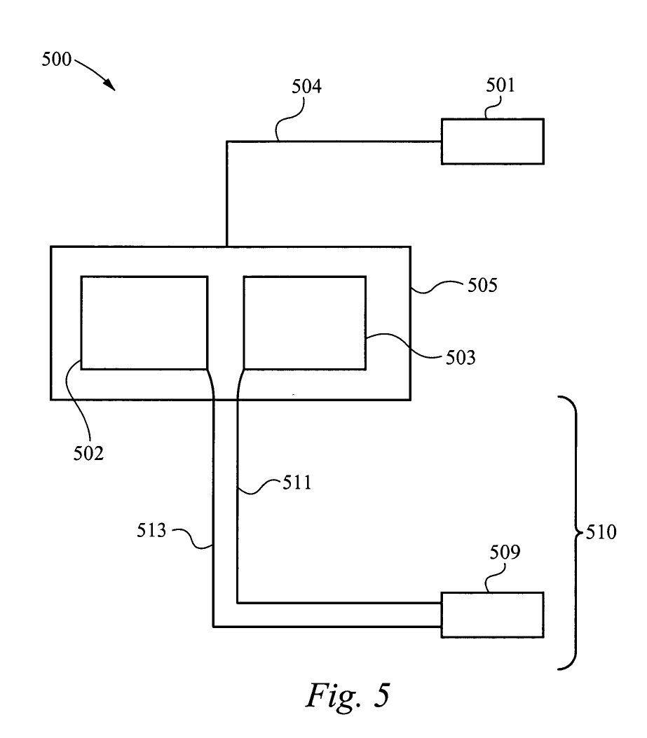

FIG. 5 shows a laser system 500 in accordance with the current invention. The laser system 500 preferably comprises a housing 505 for housing at least one laser source 502. The laser source 502 is preferably a Nd:YAG laser source. The laser source 502 is coupled to a power source 501 through the appropriate electrical connection 504 to provide power to the source 502. The laser system 500 also has a delivery system 510 for delivering laser light to a target tissue (not shown). In an embodiment of the invention delivery system 510 comprises an arm feature 513 for housing mirrors, lenses, optical fibers or any combination thereof. Alternatively, the delivery system 510 utilizes on or more optical fibers. Regardless of the optical used, the delivery system 510 is preferably configured for controllably directing laser radiation from the laser source 502 onto the target tissue. The delivery system 510 also preferably comprises an applicator 509. The applicator 509 is preferably configured to interface with the target tissue during laser treatment.

In accordance with an embodiment of the current invention, the laser system 500 also has a cooling source 503. The cooling source 503 is coupled to a cooling line 511 to deliver a cooling medium to the target tissue through the applicator 509. The cooling medium is a gas or a liquid or any combination thereof and is used to regulate the temperature of soft periodontal tissue before, during or after laser treatment.

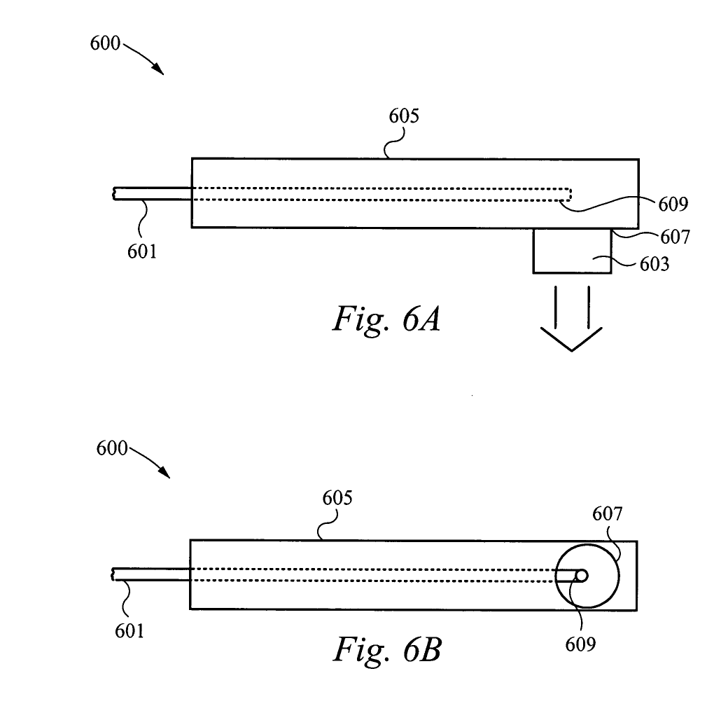

Referring now to FIGS. 6A-B, the applicator 600 of the instant invention preferably comprises an applicator housing 605 configured to be hand-held. An optical fiber 601 with a firing end 609 extends through the applicator housing 605 and is positioned to deliver laser radiation through an aperture 607 to a target tissue. The applicator 600 preferably has a guide member 603 that contacts soft periodontal tissues and regulates the distance between the firing end 609 and the target tissues during laser treatment. The guide member 603 is preferably formed from a soft resilient material, such as rubber, silicon, or latex and encircles the aperture 607. Preferably, the applicator 600 and or guide member 603 is capable of being sterilized or, alternatively, is configured to be disposable.

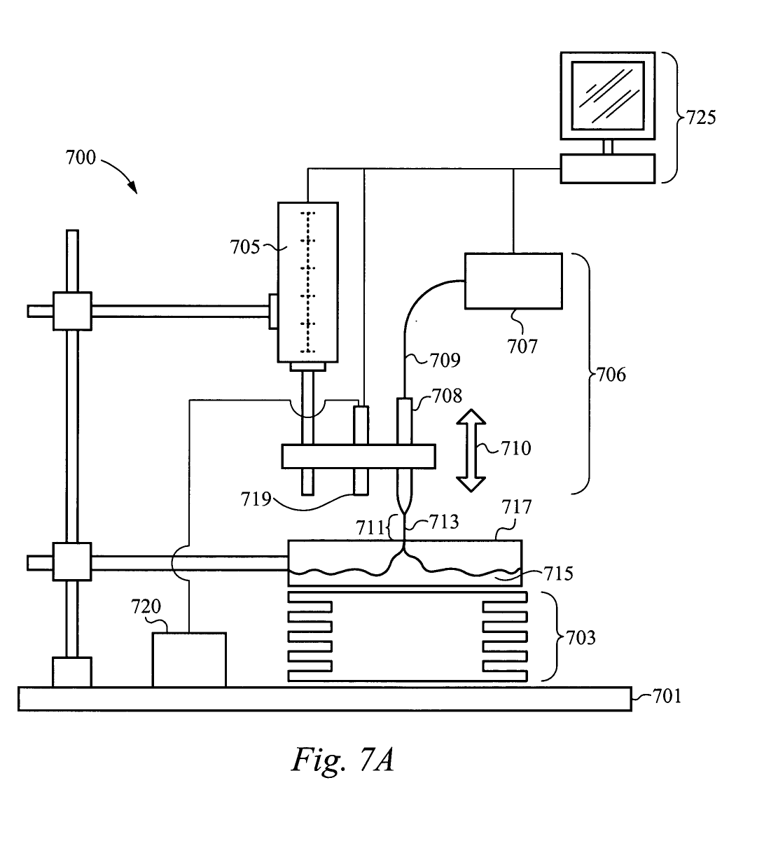

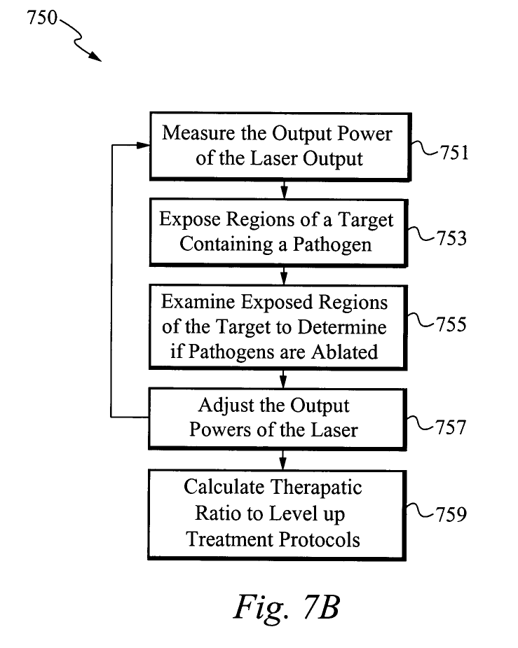

FIG. 7A shows an apparatus 700 for measuring therapeutic or antiseptic laser doses for eradicating or ablating pathogens in a target, in accordance with the embodiments of the invention. The apparatus 700 comprises an adjustable laser source 706 for generating a laser output 713. The adjustable laser source 706 preferably comprises a Nd:YAG laser 707, a trunk fiber 709 and a delivery optical fiber 708, such as described above. The adjustable laser source 706 is preferably configured to provide a range of laser exposures to a target 715. The exposures can be varied by changing the laser output power from the laser 707, changing the exposure area (the laser spot size), changing a distance 711 between the delivery optical fiber 708 of the laser source 707 and the target 715 or any combination thereof. The distance 711 between the delivery optical fiber 708 of the laser source 706 and the target 715 can be controlled with a stepper motor unit 705 that is configured to incrementally change the distance 711 between the delivery optical fiber 708 of the laser source 706 and the target 715 by moving the delivery optical fiber 708 in a direction 710.

The apparatus 700 preferably comprises means for holding the target 717 in a path of the laser output 713. The means for holding the target in a path of the laser output can include a stand 701 and dish 717 configured for holding target 715. The target 715 can include a medium, such as gelatin, and a pathogen, such as phorphyromonas gingivalis (Pg) and prevotella intermedia (Pi) and/or a pigment fungi. The apparatus 700 preferably further comprises a means for measuring the laser power provided to the target 715 from the adjustable laser source 706. For example, the means for measuring the laser power provided to the target 715 from the adjustable laser source 706 comprises a power meter 703 that is configured to be positioned with the target 715 between the power meter 703 and the path of the laser output 713.

In accordance with still further embodiments of the invention, the apparatus 700 comprises an optical detector 719, such as an optical microscope, optical scanner and/or a video camera with a recorder 720 for determining when exposed pathogens within the target have been eradicated or ablated by the laser exposure and/or the percentage of exposed pathogens that are eradicated or ablated by a laser exposure. The apparatus 700 can also include an audio detector (not shown) for detecting clicks or sounds generated while exposing the target 715 to the laser radiation.

The apparatus 700, in accordance with further embodiments of the invention, has a computing means 725. The computing means 725 is coupled to the adjustable laser source 706, power meter 703, the optical detector 719, the stepper motor unit 705 or any combination thereof. The computing means 725 can be interfaced with the adjustable laser source 706, the power meter 703, the optical detector 719, the stepper motor unit 703 or any combination thereof, to automatically collect and store data acquired during an exposure of the target 715 with laser radiation from the adjustable laser source 706. Data that are collected and stored by the computing means 725 can include data related to laser output power levels, exposure times, exposure spot sizes, laser repetition rates, percentages of pathogens ablated in the laser exposed areas of the target 715, distances 711 between laser output 713 and the target 715, etc. Accordingly, multiple pathogen targets and/or samples can be treated under a wide range of laser exposure conditions to provide a statistically significant amount of data that can then be analyzed and used to develop laser treatment protocols for treating periodontal tissue infected, for example, with the pathogens.

FIG. 7B shows a block diagram 750 outlining steps for calculating a therapeutic or antiseptic laser dose for eradicating or ablating pathogens in a target, in accordance with the method of the present invention. The antiseptic dose for a pathogen in a target can be determined by measuring the power of a pulsed laser output from a laser source in the step 751. After measuring the output power of a pulsed laser in the step 751, regions of a target containing the pathogens are exposed with the pulsed laser output in the step 753. After the regions of a target containing the pathogens are exposed with the pulsed laser output in the step 753, the exposed regions of the target are examined in the step 755 to determine if the pathogens within the exposed regions of the target have been eradicated or ablated. If in the step 755 a significant percentage (75 percentage or more) of the pathogen have been eradicated or ablated by laser exposure, then the output power of the laser source is reduced in the step 757 and exposure steps 751, 753 and 755 are repeated in new regions of the target. If in the step 755 a significant percentage of the pathogens have not been eradicated or ablated in the exposed areas of the target, then in the step 757 the laser output power is increased and the exposure steps 751, 753 and 755 are repeated in new regions of the target. By an iterative process, such as described above, a range of exposure conditions that are capable of eradicating a significant percentage of the pathogens within laser exposed regions of the target can be determined and laser therapies developed in the step 759. In accordance with a preferred method of the invention the laser source is pulsed at a repetition rate corresponding to a photo-acoustic frequency of the of the target to determine the lowest level of laser energy required to eradicate or ablate the pathogens. When the laser source is pulsed at the photo-acoustic frequency of the target an audible click is produced which can be monitored by an audio detector, such as described above with reference to FIG. 7A.

After the therapeutic or antiseptic dose is determined, then based on knowledge of tissue responses to similar laser radiation exposures, therapeutic protocols or models for treating periodontal tissues infected with the pathogen can be developed. A detailed description of tissue responses, dosimetry and laser response curves for calibrating lasers is provided in U.S. Provisional Patent Application, Serial No. 60/464,929, filed Apr. 22, 2003, and titled “METHOD OF PERIODONTAL LASER TREATMENT”, referenced previously.

The current invention provides a system for and a method of developing protocols for the treatment of periodontal tissues infected with pathogens. The system and method can be generic equipment that provides general models and guidelines for periodontal laser therapy or can be specific equipment that provides guidelines for operating specific equipment for periodontal laser treatment. The system can be automated to catalog responses of tissues, pathogens and combinations thereof under a large number of laser exposure conditions, providing invaluable information to both manufactures of laser equipment and practitioners alike.

The present invention has been primarily described with reference to its application in the treatment of periodontal disease. However, it will be clear to one skilled in the art that the treatment of periodontal disease is used herein as a model for developing treatment protocols and that the system and method of the present invention can be used to treat any number of diseases. that involve pigmented pathogen that colonizes on a luminal surface, such as pathogens associated with cystic fibrosis, topological fungi and the like. Further, laser treatments, such as described herein, can be performed on any part of a human or an animal where a delivery optical fiber can be positioned to irradiate target pathogens, such as nasal cavities, sinus passages, intestinal tracks, ear canals and throat passages, to name a few.

What is claimed is:

1. An apparatus for determining an antiseptic laser dose, the apparatus comprising:
a) an adjustable laser source for generating a laser output;
b) means for holding a target in a path of the laser output; and
c) means for measuring a laser power from the adjustable laser source,
wherein the antiseptic laser dose is determined by ablation of exposed pathogens within the target.

2. The apparatus of claim 1, wherein the adjustable laser source comprises a pulsed laser for generating pulsed laser light having a wavelength in a range of 580 to 1800 nanometers.

3. The apparatus of claim 2, wherein the pulsed laser is a Nd:YAG laser.

4. The apparatus of claim 3, wherein the adjustable laser source comprises an optical fiber configured deliver the laser output to the target with an exposure area in a range of 1.0 to 9 mm².

5. The apparatus of claim 1, wherein the adjustable laser source comprises means for controlling a distance between the laser output and the target.

6. The apparatus of claim 5, wherein the means for controlling the distance between the laser output and the target comprises a stepper-motor unit.

7. The apparatus of claim 1, further comprising means for measuring the ablation of the exposed pathogens.

8. The apparatus of claim 7, wherein the means for measuring the ablation of the exposed pathogens comprises an optical microscope.

9. The apparatus of claim 8, wherein the optical microscope is configured to optically measure a percentage of the exposed pathogens that are ablated in an area of the target.

10. The apparatus of claim 1, wherein the means for measuring the laser power from the adjustable laser source comprises a power meter.

11. The apparatus of claim 10, further comprising a means for holding the target between the path of the laser output and the power meter.

12. The apparatus of claim 1, further comprising means for determining a therapeutic radiation for treating a periodontal tissue hosting the pathogen.

13. A method of determining a damage threshold for delivering an antiseptic dose to a pathogen in a target, the method comprising:
a. measuring a pulsed laser output from a laser source;
b. irradiating the target with the pulsed laser output, wherein the target comprises the pathogen;
c. examining the pathogen for ablation;
d. adjusting the pulsed laser output; and
e. repeating steps (a) through (d) to determine the ablation threshold of the pathogen within the target.

14. The method of claim 13, wherein adjusting the pulsed laser output comprises controlling a distance between a firing end of the laser source and a surface of the target.

15. The method of claim 13, wherein the pulsed laser output is delivered at a repetition rate corresponding to a photo-acoustic of the target.

16. The method of claim 13, further comprising calculating a therapeutic ratio for treating a periodontal tissue comprising the pathogen.

17. The method of claim 16, further comprising selecting a treatment protocol for treating periodontal tissues that host the pathogen based on the therapeutic ratio.

18. The method of claim 13, wherein the pulsed laser output corresponds to a wavelength in a range of 580 to 1800 nanometers.

19. The method of claim 13, wherein irradiating the target with the pulsed laser output comprises exposing the target through an optical fiber.

20. The method of claim 13, wherein examining the pathogen for ablation comprises scanning an exposed region of the target with an optical scanning means.

21. A system for eradicating bacteria colonized within periodontal tissue, the system comprising a detachable endo-probe configured to deliver pulsed laser radiation having a wavelength in a range of 580 to 1800 nanometers to the periodontal tissue, wherein the endo-probe comprises a means for measuring depths of periodontal pockets in the periodontal tissue.
